(19) United States
(12) Patent Application Publication
(10) Pub. No.: US 2005/0175715 A1
(43) Pub. Date: Aug. 11, 2005

(54) Cellular depolarization and regulation of matrix metalloproteinases

(76) Inventors: Stephen H. Monroe, Memphis, TN (US); Robert M. Moore II, Nesbitt, MS (US)

Correspondence Address: O'MELVENY & MEYERS, 114 PACIFICA, SUITE 100, IRVINE, CA, 92618, US

(21) Appl. No.: 10/925,733

(22) Filed: Aug. 25, 2004

Related U.S. Application Data
(60) Provisional application No. 60/497,600, filed on Aug. 25, 2003.

(51) Int. Cl. A61K 31/505; A61K 31/44; A01N 33/12; A61K 31/14

(52) U.S. Cl. 424/722; 514/269; 514/352; 514/456; 514/642

(57) ABSTRACT

A mechanism is disclosed wherein depolarization of the cells associated with disease states, wherein matrix metalloproteinases are implicated as a contributor to the pathology of the disease state, and the subsequent regulation of certain proteins beneficially aids control of the matrix metalloproteinases. This initiating trigger can ultimately result in the down-regulation or up-regulation of matrix metalloproteinases. An example is given wherein matrix metalloproteinase 2 (MMP 2) is down-regulated.

### CROSS-REFERENCE TO RELATED APPLICATIONS

This application is a non-provisional application based on Provisional application Ser. No. 60/497,600, filed Aug. 25, 2003, entitled: Cellular Depolarization and Regulation of Matrix Metalloproteinases, the entirety of which is incorporated herein and upon which priority is claimed.

### STATEMENT REGARDING FEDERALLY SPONSORED RESEARCH OR DEVELOPMENT

Not Applicable

### FIELD OF INVENTION

This invention relates to methods and compositions useful in the treatment of cells associated with disease states wherein matrix metalloprtoteinases are implicated as a contributor to the pathology of the disease state.

### BACKGROUND OF INVENTION

The imbalance of ion concentrations on each side of the cell membrane of a living being defines the electrochemical gradient that is essential for cellular function. At this point cellular function is only defined as maintaining the viability of a particular cell type and does not reflect “normal” function. The electrochemical gradient maintained by all cells is essential for cellular homeostasis.

### BRIEF DESCRIPTION OF FIGURES

FIG. 1 is a representation of an electrochemical gradient across a cell membrane;

FIG. 2 is a diagramtic representation of the electrochemical transport of potassium cations across a cell membrane, the opening of a K⁺ channel as a consequence of intracellular and/or extracellular signaling via biochemical or synthetic chemical activation or in response to electrochemical events, and followed by closure of the K+ channel, all as a function of the extracellular and intracellular concentrations of the potassium cations associated with a cell and its membrane;

FIG. 3 is a diagramtic representation of the extracellular and intracellular cationic concentrations of potassium, rubidium, calcium and zinc cations of a cell and the addition of a depolarizer of the present invention to the cell environment; and

FIG. 4 is a graph depicting the change, over time, of the concentration of matrix metalloproteinase in the presence of various cell membrane depolarizing agents.

### SUMMARY OF INVENTION

A mechanism is disclosed wherein depolarization of the cells in tissues or circulatory cell types, or cells associated with disease states, wherein Matrix MetalloProteinases (MMPs) are implicated as a contributor to the pathology of the disease state, results in the subsequent regulation of certain proteins, the regulation of which beneficially aids various biochemical processes affecting such disease state. This initiating trigger ultimately results in the regulation of matrix metalloproteinases 2 and/or 9 (MMP-2 and MMP-9) and/or other MMPs. Based upon current research, it appears and is proposed by the present inventors, that a membrane chemical entity, or an ionic composition such as calcium, sodium, potassium or other ionic entity in a pharmaceutically active formulation acts as a cell membrane depolarizing agent in those cells, with resultant discouragement of the deleterious production of MMPs. It is furthermore proposed that the use of any cellular membrane depolarizing agent would have a similar beneficial effect on these cells and cell types and MMP regulation.

### DETAILED DESCRIPTION OF INVENTION

Membrane Potential, Osmotic Strength, and DerMax™ Cations.

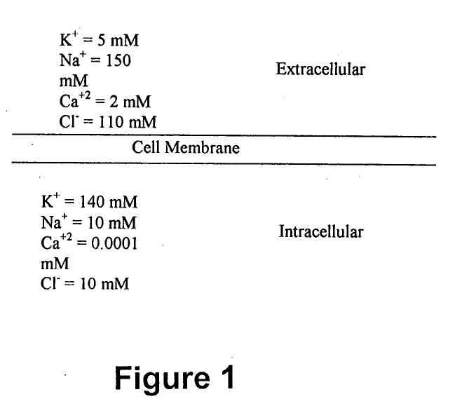

The fundamental basis for the mechanism of action of DerMax™, a treatment comprising K⁺, Rb⁺, Ca⁺², and Zn⁺², initially in the form of their chloride, resides in the electrochemical gradient maintained by all cells. This gradient is essential for cellular homeostasis and is achieved by partitioning different concentrations of K⁺, Na⁺, Ca⁺², and Cl⁻ ions on the intra- and extracellular sides of the cell membrane (FIG. 1).

The imbalance of ion concentrations on each side of the cell membrane defines the electrochemical gradient that is essential for cellular function. At this point cellular function is only defined as maintaining the viability of a particular cell type and does not reflect “normal” function. Furthermore, from this point forward herein, the effects of Dermax™ on the gradient will not be based on a rigorous physical chemical treatment, ie., based on the Nernst equation, the Goldman equation, and Ohm's Law. Instead, for the sake of clarity, the effects of DerMax™ on the said cells will be based on the equilibrium of the system.

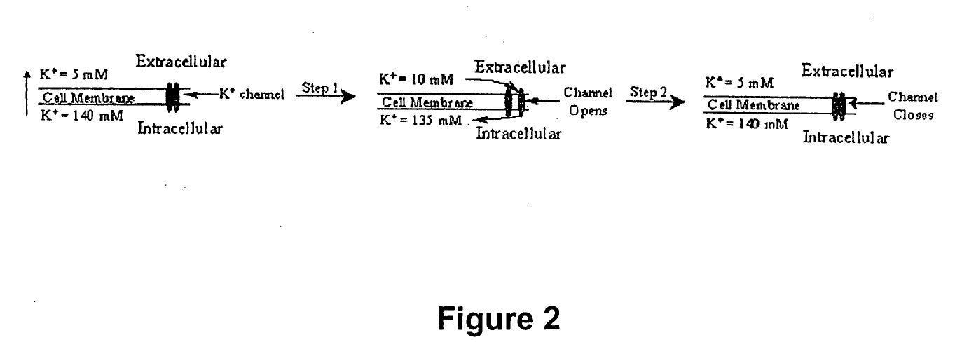

Under normal conditions the cell membrane controls the influx and outflux of is permeable to K⁺, Na⁺, and Cl⁻ and other select elemental ions. Of these ions, K⁺ has a much higher permeability to the cell membrane than Na⁺, and Cl. The net effect is that the K⁺ concentration on the exterior and interior of the cell approximately represents the equilibrium electrochemical potential of the cell. Clearly a discontinuity exists, if the membrane is leaky to K⁺ then no K⁺ gradient could be established and as such the potential of the cell could not be approximated by the gradient of K⁺. The answer to the paradox is the evolution of protein that can shuttle ions between the extracellular and intracellular matrix, ie., the Potassium ion (K⁺) channels (See FIG. 2).

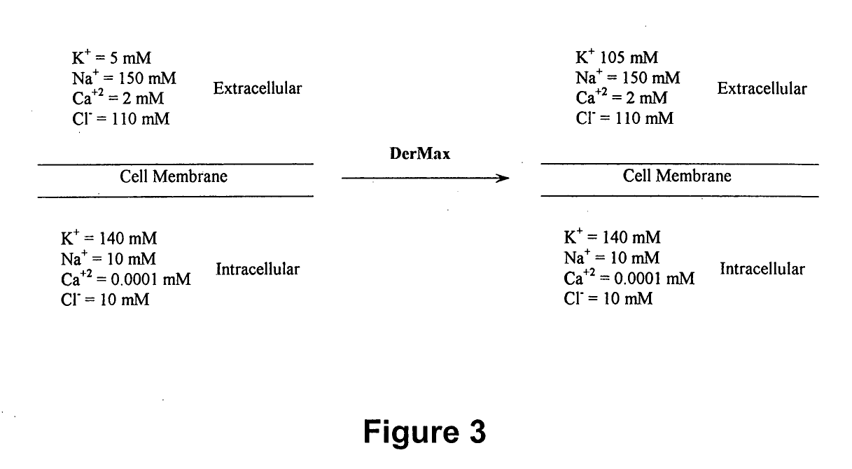

In this case, inward flowing, ‘inward rectifying’, channels allow the influx of K⁺ into the cell. In this model the leakage of K⁺ from the cell results in re-equilibration via inward rectifying K⁺ channels (Kᵢᵣ). The modest leakage of K⁺ in this example, i.e. based on a molar concentration, is not comparable to the probable effects of DerMax™ on the gradient of the cell. The potentially high K⁺ concentrations experienced by the cells associated with disease states wherein MMPs are implicated to contribute to the pathology of the disease state would abolish the K⁺ gradient resulting in the initial depolarization of the cell by negating the K⁺ gradient, i.e. increasing the extracellular K⁺ to +100 mM (see FIG. 3).

FIG. 3 intentionally neglects the extracellular concentrations of Rb⁺, Ca⁺², and Zn⁺² because their concentrations in DerMax™ are negligible relative to the gradient; furthermore, in the case of Rb⁺ and Zn⁺² these ions are not associated with maintenance of the membrane potential. The application of extracellular K⁺ under the conditions of normal intracellular K⁺ effectively abolishes the electrochemical gradient resulting in depolarization of the cell. Specifically, the cell cannot compensate for the change in the ionic gradient via K⁺ channel activation. The resulting depolarization of the cells will result in the alteration of the phenotype of the associated cells. This change in phenotype expresses itself as the down-regulation of non-essential enzymes, specifically MMP-2 and/or MMP-9 as well as others, and the up-regulation of other enzymes or proteins, such as membrane channels.

The concept of utilizing cellular depolarization is not restricted to the application of high concentrations of K⁺ or any other ion. In fact a diverse family of organic molecules are capable of depolarizing the cell membrane and are used clinically for treating disease states ranging from anti-arrhythmic agents to hair growth stimulants. In the context of the present invention, any agent that depolarizes the cells has application to the therapeutic end-point of regulating MMPs.

Potassium Channels and Novel Therapies for Disease Therapy.

The discovery of an ionic therapy for MMP regulation represents a milestone in the genesis of novel chemotherapeutic options. It is presently believed that this concept is extendable to the use of chemical entities that regulate the electrochemical gradient of cells. Based on the composition of DerMax™ it is proposed that alteration of K⁺ channel activity, as previously noted, provides this novel area for therapeutic development.

The K⁺ family of proteins can be broadly grouped into four subclasses that include 53 voltage dependent channels, 10 calcium activated channels, 17 inward rectifying channels, and 14 background channels. Other channels susceptible to depolarization would result in similar effects.

Regulation of Protein Expression.

The alteration of membrane potential can result in a number of changes in protein expression. It is anticipated that this change in protein production would similarly affect other non-essential, aberrant proteins and enzymes directly associated with different disease states.

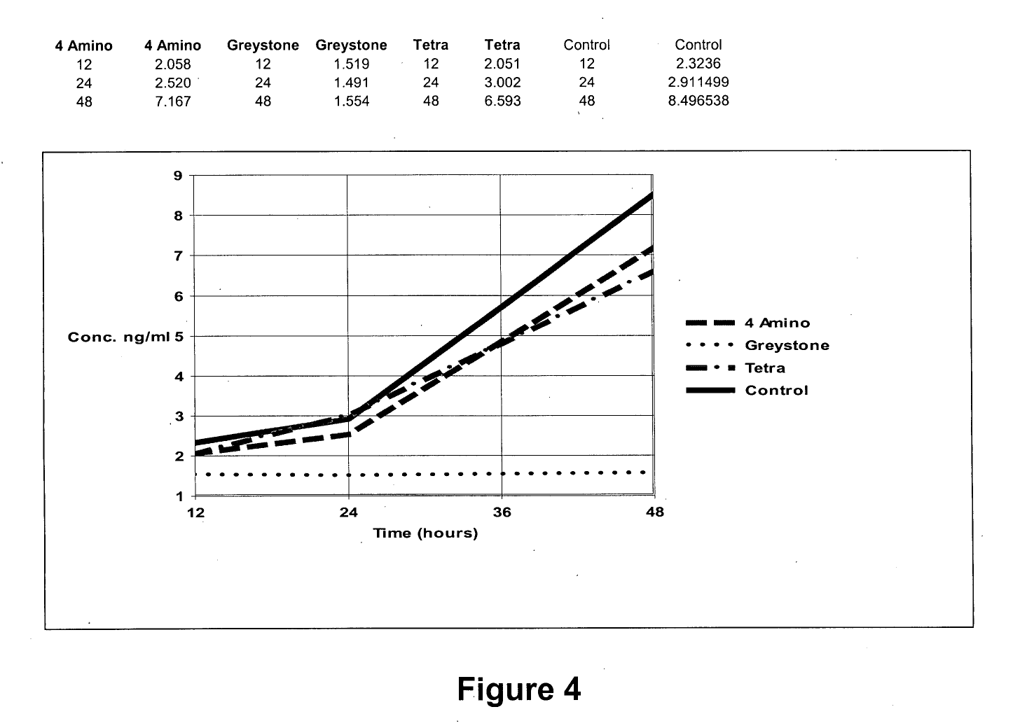

FIG. 4 depicts the change in concentration of MMP-2, over time, in cells aberrantly expressing this MMP when treated with a variety of membranes depolarizers. In FIG. 4, “4 Amino” is 4-amino-pyridine, “Greystone” is a composition in accordance with the present invention containing effective amounts of K⁺ (ie. DerMax™), “Tetra” is Tetra butyl ammonium chloride, and “Control” is growth media only. From FIG. 4 it will be noted that each depolarizer effected an initial lowering of MMP-2, but after about 24 hours, the concentration of MMP-2 increased dramatically for the control and less so for all of the depolarizers except “Greystone”. With respect to the depolarizer of the present invention, the level of MMP-2 in the cells initially lowered the concentration of MMP-2 in the cells to a level lower than the other depolarizers, and importantly, this lowering effect continued constant over the 48 hour test period.

1. A method of treatment of cells associated with disease states wherein Matrix MetalloProteinases are implicated as a contributor to the pathology of the disease state wherein the method includes a chemical entity that alters or effects the permeability of cellular membranes and calcium, sodium, potassium or other cationic or anionic ionic channels in living beings comprising the steps of introducing to said cells a pharmaceutically effective amount of a depolarizer for the cell membrane of said cells, thereby establishing an environment within said cells which modulate the formation within, or transport out of, said cells of at least one matrix metalloproteinase and concomitantly establishes an environment within said cells which is conducive to favorable ionic transfer across the cell membrane and resultant homeostasis of the cell membrane.

2. The method of claim 1 wherein said modulation of said matrix metalloproteinases comprises MMP-2 or MMP-9.

3. The method of claim 1 wherein said modulation comprises discouragement of the formation within, or transport out of, said cells of at least one matrix metalloproteinase and concomitantly establishes an environment within said cells which is conducive to favorable ionic transfer across the cell membrane and resultant homeostasis of the cell membrane.

4. The method of claim 1 wherein said modulation comprises encouragement of the formation within, or transport out of, said cells of at least one matrix metalloproteinase and concomitantly establishes an environment within said cells which is conducive to favorable ionic transfer across the cell membrane and resultant homeostasis of the cell membrane.

5. A method of ionic therapy for MMP regulation within living cells wherein the osmolality of the cellular environment is changed by addition of ionic species in solution to effectively abolish the electrochemical gradient across the cell membrane and resultant depolarization of the cell membrane and establishment of an environment conducive to the modulation of the formation within, or transport out of, the cells of at least one matrix metalloproteinase.

6. The method of claim 3 wherein said extracellar cations include, but are not limited to, a pharmaceutically effective amount of a solution of extracellular cations, including at least one of potassium, rubidium, calcium and zinc cations, to effectively abolish the electrochemical gradient across the cell membrane and resultant depolarization of the cell membrane and establishment of an environment deleterious to the formation within, or transport out of, the cells of matrix metalloproteinases.

7. The method of claim 3 wherein said extracellar cations are potassium.

8. The method of claim 3 wherein said potassium cations are present in said solution in a concentration of between about 0.1 and about 5 percent, by weight.

9. The method of claim 1 wherein said depolarizer of the cell membrane of cells is a known membrane depolarizer.

10. The method of claim 9 wherein said depolarizer of the celllmembrane comprises 4 amino pyridine, tetra butyl ammonium chloride, minoxidil, apomin, cromakalim and combinations thereof.

11. A method of regulation of the electrochemical gradient of cells of living beings comprising the step of introducing into the cell environment a solution containing a chemical entity effective to modulate transfer of selected ionic entities across the cell membrane.
